(19) United States
(12) Patent Application Publication
(10) Pub. No.: US 2012/0111743 A1
(43) Pub. Date: May 10, 2012

(54) BINARY FOAMING CLEANER AND DISINFECTANT SOLUTION

(75) Inventors: Shira Kramer, Timonium, MD (US); Mark Wozniak, Bel Air, MD (US)

(73) Assignee: Sterilex Corporation, Hunt Valley, MD (US)

(21) Appl. No.: 13/321,573

(22) PCT Filed: May 27, 2010
(86) PCT No.: PCT/US2010/036432

§ 371 Date: Jan. 19, 2012

Related U.S. Application Data
(60) Provisional application No. 61/217,108, filed on May 27, 2009.

(51) Int. Cl. B65D 25/08; A61K 9/12

(52) U.S. Cl. 206/219; 424/44

(57) ABSTRACT

A binary composition is provided comprising two liquids, which are separately maintained prior to forming an admixture during delivery to a hard surface, vessel, or drain. The admixture generates foam of sufficient stability and quantity to effect disinfection and biofilm removal. A first liquid preferably includes a quaternary ammonium compound, a peroxygen agent and, optionally, surfactants, thickening and chelating agents (colorants, corrosion inhibitors, stabilizers, perfume, etc). The second liquid preferably includes a hypohalite generating agent, a source of alkalinity, and optionally, a surfactant and thickening and chelating agents (colorants, corrosion inhibitors, stabilizers, perfume, etc.). Because the two liquids are initially separated, the hy-pohalit and peroxygen agent can be maintained in an environment that is favorable to their stability until the time of use. When the two liquids are mixed, the hypohalite and peroxygen react to liberate oxygen gas. Foam generation occurs as the escaping gas contacts the quaternary ammonium compound and surfactants and creates foam, which expands to completely fill or cover the drain pipe, or other vessel or a hard surface being treated. The expanded foam contains quaternary ammonium compound and hydrogen peroxide not reacted with the hypohalite generating agent, which act to disinfect, clean and remove biofilm

### BACKGROUND OF THE INVENTION

1. Field of the Invention

The present invention relates to foaming compositions and, in particular, to an in-situ created foaming disinfectant composition incorporating a quaternary ammonium and an alkaline peroxygen compound, which is formulated to have utility as a disinfectant, cleaner, and biofilm remover in drains or on other hard surfaces and vessels.

2. Background of the Prior Art

Disinfection of hard surfaces, drains, trunk lines, and vessels in private and public facilities, such as restaurants, food-processing facilities, grocery stores and big box retailers, is essential to prevent bacterial contamination and cross contamination. Likewise, surfaces in kitchens, hospitals, hospital floors and drains, bathroom/shower facilities, and many other facilities are also a source of microorganisms that require control.

A 2004 audit of food processing facilities inspected by the USDA Food Safety and Inspection Service reported that 27.8% of floors and drains inspected tested positive for Listeria monocytogenes, a dangerous food pathogen (Buege & Ingham, 2004, Audit of Post-Lethality Environment of Very Small Processing Plants for Listeria Species. Madison, Wis., USA. http://www.meathaccp.wisc.edu/backgrounder/auditsummaryl.s124.pdf, last visited: May 27, 2010). In food preparation areas, bacteria in drains is often identified as the prime source of cross-contamination causing food poisoning and product recalls. The Food and Drug Administration has cited flies as a disease-carrying insect that transmits bacteria such as Staphylococcus, E. coli, and Salmonella every time they land. Further exacerbating the danger of unsanitary drains is the presence of flies, including fruit flies, which live off of the debris, slime, and bacterial biofilms attached to the walls of the drain. (http://www.proflycontrol.com/page/page/1210200.htm.) Microbial contamination from drains in hospital settings has been shown to be associated with a higher risk of hospital-induced infections and even death in hospitals (Melnbardis, 2007, Dirty Hospital Drains Blamed in Canada Baby Deaths. From CareConnection.com. http://www.healthcentral.com/caregiver/news-37866-66.html, last visited: May 27, 2010; and Hota, S. et al., 2009, Outbreak of multidrug-resistant Pseudomonas aeruginosa colonization and infection secondary to imperfect intensive care unit room design, Infect Control Hosp Epidemiol 30:25-33, last visited May 27, 2010.) The microbial contamination is difficult to control and the associated pathogens are often resistant to antimicrobials because of biofilm formation in the drains and on other hard surfaces.

Biofilms are microbial (bacterial, fungal, algal) communities, enveloped by an extracellular biopolymer, which these microbial cells produce, that adheres to the interface of a liquid and a surface. An adhesive substance, the glycocalyx, envelops the bacterial community at the interface of a liquid and a surface. When a liquid is in contact with an inert surface, any bacteria within the liquid are attracted to the surface and adhere to it. Microbial fouling or biofouling are the terms applied to these actual or potentially undesirable consequences. (http://www.answers.com/topic/biofilm, last visited on May 12, 2010, Biofilms in Infections. From http://www.wiziq.com/tutorial/40211-Biofilms-in-Infection, last visited: May 27, 2010.)

Microbial fouling affects a large variety of surfaces under various conditions. Microbial biofilms may form wherever bacteria can survive. Biofilms can form on any hard surface, including, for example, medical equipment. The CDC (Centers for Disease Control) estimates that over 65% of nosocomial (hospital-acquired) infections are caused by biofilms (Rao, 2010). Bacteria growing in a biofilm are highly resistant to antimicrobials, up to 1,000 times more resistant than the same bacteria not growing in a biofilm. Standard antimicrobial/antibiotic therapy is often useless and, in cases where a medical device is implanted, the only recourse may be to remove the contaminated implant. Fungal biofilms also frequently contaminate medical devices. (http://www.medterms.com/script/main/art.asp?articlekey=16932, last visited May 12, 2010.)

Disinfectants are frequently used in hospitals, during dental surgeries, in kitchens, and in bathrooms to kill or otherwise control pathogenic or infectious organisms. Many types of disinfectants are known. However, all have inherent limitations and are typically used only in specific circumstances.

Among known disinfectants occasionally used to disinfect hard surfaces and pipes or drains are aldehydes, such as formaldehyde and glutaraldehyde. They are partially inactivated by organic matter and have a slight residual activity. Some bacteria have developed resistance to glutaraldehyde, and it has been found that glutaraldehyde can cause asthma and other health hazards (http://en.wikipedia.org/wiki/Disinfectant, last visited May 10, 2010). In addition, glutaraldehyde is a cross-linking agent, and when used on a biofilm, will cause the biofilm to become more resistant to removal.

Oxidizing agents are commonly used disinfectants. Chlorine and oxygen are examples of oxidizers. Typically, instead of chlorine, chlorine-containing compounds are used. Examples include sodium, calcium or bromide hypochlorite or chlorine dioxide releasing compounds such as sodium chlorite, sodium chlorate and potassium chlorate. The high levels of oxidizing agents needed to “burn off” biofilms can be corrosive.

Quaternary ammonium compounds (“QAC”) are also used as disinfectants. However, they are not very effective at penetrating biofilms and killing micro-organisms residing in the biofilms and typically dry off quickly, reducing their efficacy. Id.

Clearly, additional/alternative disinfectant solutions are desirable.

### SUMMARY OF THE INVENTION

The invention provides in accordance to one aspect an in-situ foaming aqueous composition for disinfecting a drain, a vessel, or a hard-surface comprising at least two liquid solutions mixing in-situ and creating the foaming composition, which foaming composition comprises a quaternary ammonium compound, peroxygen, and is at an alkaline pH,
wherein, premixing, at least one of the liquid solutions comprises water and a peroxygen,
wherein, premixing, at least one of the liquid solutions comprises water, a hapohalite and an alkaline pH adjusting agent, and
wherein, premixing, at least one of the mixing water solutions solution comprises a quaternary ammonium compound.

In accordance to one embodiment the hypohalite is a hypochlorite. A preferred hypochlorite is a sodium hypochlorite.

In accordance to another embodiment, the peroxygen compound is selected from among hydrogen peroxide, a perborate, a percarbonate, an organic peracid, an inorganic peracid or a persalt. Preferably, the peroxygen compound is hydrogen peroxide.

In accordance to yet another embodiment, the pH adjusting agent is a carbonate, bicarbonate, hydroxides, or oxide. Preferably, the pH adjusting agent is a sodium salt of a carbonate, bicarbonate, hydroxides, or oxide or potassium salt of carbonate or calcium salt of oxide or hydroxide.

In accordance to still another embodiment, the molar basis the peroxygen is in excess of the hypohalite in the solutions premixing and the foaming composition comprises about between 100 and 50,000 ppm peroxygen. Preferably, the foaming composition comprises about between 650 and 50,000 ppm peroxygen.

In accordance to still yet another embodiment, the quaternary ammonium compound in the foaming composition is at about between 100 ppm and 50,000 ppm.

In accordance to a further embodiment, the foaming composition comprises between about 650 ppm to 50,000 ppm of the quaternary ammonium compound and about between 650 ppm to 50,000 ppm of the peroxygen and the composition is a biofilm remover.

In accordance to a further yet another embodiment, at least one of the two liquid solutions further comprises at least one of a chelating agent, a surfactant, a thickening agent, a perfume, a corrosive inhibitor. Preferably, the composition further comprises a chelating agent. Yet more preferably, the chelating agent is EDTA.

In accordance to a further still another embodiment, at least one of the two liquid solutions further comprises a surfactant or thickener. Preferably, the surfactant or thickener is an amine oxide, a C14-C18 alkyl betaine, an alkyl glucoside or a nonionic surfactant or is a surfactant.

In accordance to another aspect, the invention provides a container for maintaining at least two liquids separately, comprising at least a first compartment for storage of a first liquid and a second compartment for storage of a second liquid, where the first liquid comprises a hypohalite and the second container comprises a peroxygen, wherein the liquid from each container may be exuded from the respective compartment via separate delivery channels that either
allows for the mixing of the liquids in a chamber prior to the delivery of the mixture from the container, or
delivers the liquids such as no mixing of the liquids occurs prior to their exit from the container.

In accordance to yet another aspect, the invention provides a method to prepare in-situ a foaming disinfectant and aqueous composition comprising
providing at least one solution comprising water and a peroxygen; providing at least one solution comprising water, a hapohalite and an alkaline pH adjusting agent; providing at least one solution comprising water and a quaternary ammonium compound,

wherein the solution comprising peroxygen can not comprise hapohalite,

wherein the solution comprising a quaternary ammonium compound may be a separate solution or may be the same solution that comprises a peroxygen or the same solution that comprises a quaternary ammonium compound,
mixing the above solutions in a pipe, vessel or a hard surface, whereby foam is formed in-situ, and
treating to disinfect the pipe, vessel or a hard surface by exposure to the foam solution for at least two minutes, and wherein the foaming solution is at least twice the combined volumes of the liquid solutions premixing, and ‘

wherein the foaming solution comprises a quaternary ammonium compound, peroxygen, and is at an alkaline pH, and

wherein the foaming solution disinfects the pipe, vessel or a hard surface.

In accordance to still another aspect, the invention provides method to prepare in-situ a foaming disinfectant and aqueous composition comprising:
mixing in a pipe, vessel or a hard surface at least two liquid solutions wherein, premixing, at least one of the liquid solutions comprises water and a peroxygen, wherein, premixing, at least one of the liquid solutions comprises water, a hapohalite and an alkaline pH adjusting agent,

wherein, premixing, at least one of the liquid solutions comprises water and a quaternary ammonium compound,

wherein the solution which, premixing, comprises hapohalite does not contain peroxygen, and
treating to disinfect the pipe, vessel or a hard surface by exposure to the foam solution for at least two minutes,

wherein the foaming solution is at least twice the combined volumes of the liquid solutions premixing,

wherein the foaming solution comprises a quaternary ammonium compound, peroxygen, and is at an alkaline pH, and

wherein the foaming solution disinfects the pipe, vessel or a hard surface.

In accordance to one embodiment, the hypohalite is a hypochlorite. Preferably, the hypochlorite is sodium hypochlorite.

In accordance to another embodiment, the peroxygen compound is selected from among hydrogen peroxide, a perborate, a percarbonate, an organic peracid, an inorganic peracid or a persalt. Preferably, the peroxygen compound is hydrogen peroxide.

In accordance to still another embodiment, the pH adjusting agent is a carbonate, bicarbonate, hydroxides, or oxide. Preferably, the pH adjusting agent is a sodium salt of a carbonate, bicarbonate, hydroxides, or oxide.

In accordance to still another embodiment, on a molar basis the peroxygen is in excess of the hypohalite in the solutions premixing and the obtained foaming composition comprises about between 100 and 50,000 ppm peroxygen. Preferably, the foaming composition comprises about between 650 and 50,000 ppm peroxygen.

In accordance to still yet another embodiment, the quaternary ammonium compound in the foaming composition is at about between 100 ppm and 50,000 ppm. Preferably, the foaming composition comprises between about 650 ppm to 50,000 ppm of the quaternary ammonium compound and about between 650 ppm to 50,000 ppm of the peroxygen and the composition is a biofilm remover.

In accordance to still yet another embodiment, at least one of the at least two liquid solutions further comprises at least one of a chelating agent, a surfactant, a thickening agent, a perfume, a corrosive inhibitor. Preferably, the at least one of the at least two liquid solutions further comprises a chelating agent. Yet more preferably, the chelating agent is EDTA.

In accordance to a further embodiment, the at least one of the at least two liquid solutions further comprises a surfactant or thickener. Preferably, the surfactant or thickener is an amine oxide, a C14-C18 alkyl betaine, an alkyl glucoside or a nonionic surfactant.

### DETAILED DESCRIPTION OF THE INVENTION

The invention provides a foam composition comprising hydrogen peroxide and a quaternary ammonium compound, which act synergistically under alkaline conditions to disinfect, clean and remove biofilm from a drain, vessel, or hard surface. The composition is “binary,” i.e. it is a product of at least two liquids, which am separately maintained prior to mixing. It is “binary” also in the sense that it contains at least two active ingredients, a quaternary ammonium compound and hydrogen peroxide. A first liquid typically consists of a quaternary ammonium compound and a hydrogen peroxide or hydrogen peroxide generating agent. Hydrogen peroxide and hydrogen peroxide generating agents are herein referred to also as peroxygen compounds. The second liquid typically includes a hypohalite or hypohalite generating agent and a source of alkalinity. Either liquid may optionally contain chelating agents, surfactants, colorants, and corrosion inhibitors as the particular application may require. The two liquids of the binary system are kept separated until the time of use thereby allowing the hydrogen peroxide and hypohalite compounds to be maintained in an environment suitable for their individual stability. When the two liquids are allowed to mix by the simultaneous pouring or spraying of the liquids into a drain, vessel, or hard surface, the hypohalite and hydrogen peroxide react to liberate oxygen gas in accordance with the following unbalanced equation:

Hypohalite+Hydrogen Peroxide→O₂ (gas)

The liberated gas contacts the quaternary ammonium compound and the optional surfactants in the solution creating foam, which expands to fill the drain pipe, vessel, or covers a surface area. The expanded foam preferentially contains hydrogen peroxide not reacted with the hypohalite and further contains the quaternary ammonium compound. The foam has a certain resilience/stability, allowing the disinfectant action to proceed effectively by prolonging the exposure to the active ingredients, primarily the quaternary ammonium compound and peroxide.

In accordance with a preferred embodiment, the invention comprises a stable binary disinfecting composition comprising, in aqueous solutions:
a) a first liquid consisting of at least one each of a quaternary ammonium compound and a peroxygen compound, and
b) a second liquid containing at least one hypohalite or hypohalite generating agent and an alkaline source,

wherein, upon mixing of the two solutions, the hypohalite and peroxygen compound react to generate a foam characterized by a volume of at least two times the combined liquid volumes and wherein the foam contains a effective amount of peroxygen and quaternary ammonium compound under alkaline pH to be an effective disinfectant and biofilm remover.

In accordance with an alternative embodiment, the invention comprises a stable binary disinfecting composition comprising in aqueous solutions:
a) a first liquid consisting of at least one peroxygen, and
b) a second liquid containing at least one each of a hypohalite, a quaternary ammonium compounds and alkaline sources,

wherein, upon mixing of the two solutions, the hypohalite and peroxygen compounds react to generate a foam characterized by a volume of at least two times the combined liquid volumes and wherein the foam contains an effective amount of peroxygen and quaternary ammonium compounds under alkaline pH to be an effective disinfectant and biofilm remover.

Certain variations in respect to each liquid and the presence of ingredients are possible. For example, the QAC may be present at certain levels in both solutions or stored, before mixing, separately in a third container from either the peroxygen or the hypohalite. In terms of stability of the solutions and the ability to create foam at the desired time, it is critical that the hypohalite and the peroxygen be maintained separately until about the time foam generation is desired. Preferably, one solution contains the peroxygen and the QAC, the other solution maintains the hypohalite and a source of alkalinity. The alkalinity source and the peroxygen are not desirably stored together. Although the invention description refers to the combination of at least two “liquids” or “solutions,” an artisan skilled in the art would realize that it is within the scope of the invention to combine one or more of the ingredients in a different physical form, as a solid or slurry, for example, or to combine more than two liquids and so on, as long as, upon combination, the ingredients are all in an aqueous solution.

The ratio of the first and second “liquid,” is not particularly critical. Preferentially, however, the volumes or weights of the two “liquids” are no more than about 3 fold of each other, preferably no more than about two fold, and, most preferentially they are about equal.

In another embodiment of the present invention, the first and/or second liquid also includes auxiliary thickening and surfactants necessary to impart a desired viscosity and additional foaming action to the product. The first and/or second liquid may also include other optional additives including but not limited to corrosion inhibitors, stabilizers, coloring, perfume and chelating agents.

In a preferred embodiment of the present invention, the first and/or second liquid includes a chelating agent necessary to impart hard water compatibility and to help collapse biofilm, especially in the presence of metal ions.

It is preferred that these two or more liquid compositions creating the foaming composition be stored separately until mixed together. However, it is possible to deliver one or both of the starting mixtures as powders, slurries or any physical form. Albeit the preferred embodiment is to provide a composition formed as the component mixtures are admixed, it is possible to mix the liquid compositions shortly before application or to provide the liquids successively. For example, it may be possible to deliver in a pipe the lower density mixture and follow with application of the heavier weight component mixture.

The present invention further provides a method of disinfecting drains, vessels, and other surfaces which comprises the step of pouring or spraying into a drain, vessel, or other surface liquids which generate a foam in-situ, the foam characterized by a volume of at least two times, more preferably at least about three to five times, the liquid volume and wherein the foam contains an effective amount of disinfecting active(s). Advantageously, the method and the binary composition of the two solutions also provide delivery of an effective amount of disinfecting or biofilm removing agents. “Disinfecting” means the action of destroying or killing disease causing germs or other harmful, corrosive, or spoilage microorganisms. Biofilm removal relates to arrest and dispersion of biofilm structures. Another advantage of the present invention is that the binary composition is chemically and phase-stable, and retains such stability over a wide range of temperatures. Preferably, the temperature is such that the mixture is liquid at, preferably, between −4° C. to about 60° C., more preferably from about 0° C. to about 30° C. Another advantage of the present invention is that, when formulated as a drain, vessel, or hard-surface disinfectant, the foaming composition provides a long contact time, improving the efficacy of the disinfectant/cleaner/biofilm removal composition.

The present invention also provides for a container which maintains the two liquids separately until delivery. The container includes one compartment for the peroxygen containing liquid and the second compartment contains the hypohalite containing liquid. Either or both of these two compartments may contain a thickening system and optional ingredients (e.g. chelating agents, auxiliary surfactants, corrosion inhibitors, degreasers, builders, hydrotopes, wetting agents, foam stabilizers, coloring, perfume, and other agents as might be contemplated by an artisan skilled in the art as desirable in particular circumstances). According to one aspect of the invention, the container may have separate delivery channels for the two liquid components for delivery of the two liquids, whereupon the admixture is formed. These delivery channels may be constructed to provide for the contemporaneous delivery of the two liquids to the exterior of the container, whereupon the two liquids meet to form the admixture. Alternately, the separate delivery channels may communicate with an admixing space in which the two liquids form the admixture and from which the admixture is delivered to the exterior of the container. One example of such a container is that disclosed in U.S. Pat. No. 5,767,055, Choy et al.

The composition of the invention will remain substantially in a foam phase at room temperature (5° C. to 35° C.), i.e. the foam volume will be at least about two fold the volume of the original combined liquids, premix. The foam will persist for at least about 2 minutes, preferably at least about 5 minutes and more preferably at least between about 10 minutes and 20 minutes. A longer lasting foam composition can be made by the methods and compositions of the invention and it is often desirable to create a longer lasting foam composition.

In accordance to the invention, the hypohalite is preferably selected from the group consisting of alkali metal and alkaline earth salts of hypohalite, haloamines, haloimines, haloimides and haloamides. All of these are believed to produce hypohalous bleaching species in situ. As used herein, the term “hypohalite” is used to describe both a hypohalite and hypohalite generator, unless otherwise specified. Hypochlorite and compounds producing hypochlorite in aqueous solution are preferred, although hypobromite is also a suitable hypohalite. Representative hypochlorite producing compounds include sodium, potassium, lithium and calcium hypochlorite, chlorinated trisodium phosphate dodecahydrate, potassium and sodium dichloroisocyanurate and trichlorocyanuric and tribromo-cyanuric acid, dibrom- and dichlorocyanuric acid and potassium and sodium salts thereof, N-brominated and N-chlorinated succinimide, malonimide, phthalimide and naphthalimide. Also suitable are hydantoins, such as dibromo and dichloro dimethyl hydantoin, chlorobromodimethyl hydantoin, N-chlorosulfamide (haloamide) and chloramine (haloamine). Particularly preferred in this invention is sodium hypochlorite (NaOCl) in an amount ranging from about 0.01 weight percent to about 15 weight percent of the liquid component in which it resides before the creation of the mixture composition. Other hypohalites or hypohalite generators should be used in amounts that provide the same molarity as that described here for the preferred embodiment with NaOCl.

The hypohalite may be present in a stoichiometric amount to the peroxygen for the generation of foam. A “stoichiometric amount” or “stoichiometric ratio,” of a reagent is where all reagent is consumed, no residues remain, but there is no shortfall of reagent either. When the yield is below 100%, a residue remains. If essentially all of the hydrogen peroxide is used to generate foam and no peroxide remains to act as an active ingredient, the composition will still act as a disinfecting composition but will have limited effectiveness as a biofilm remover. More preferred is that the hydrogen peroxide be present in excess, to both generate foam and synergistically combine with the quaternary ammonium compound to provide disinfection and biofilm removal.

Preferably, part of the hydrogen peroxide reacts with the hypohalite to generate a gas. The remainder of the hydrogen peroxide acts synergistically with the quaternary ammonium compound to provide disinfection and biofilm removal properties to the composition.

As used herein, the term “peroxygen” is used to describe both hydrogen peroxide and a hydrogen peroxide generator, unless otherwise specified. The preferred peroxygen compound is hydrogen peroxide, or a peracid or persalt, including both organic and inorganic peracids and persalts, such as peracetic acid and monoperoxysulfate, respectively. Perborates and percarbonates are also suitable for the composition of this invention. High concentrations of peroxygen compounds are possible in powder formulations and may be as high as 100%. Peroxygen concentrations in liquid formulations of nearly 100% would be possible theoretically but from a practical standpoint peroxygen concentrations less than about 10% are more likely practiced.

In a preferred embodiment, the amount of hydrogen peroxide in one of the solutions is present in about between 0.01 to 8 weight percent of the liquid in which it resides, pre-final mixture, preferably about between 0.5 to 7.5 weight percent, most preferably about between 1 to 7 weight percent. In embodiments where the pre-mix solution comprises a peroxygen other than hydrogen peroxide, the amount of peroxygen used should provide an equivalent amount of hydrogen peroxide on a molar basis to the above preferred embodiment using hydrogen peroxide.

Where hydrogen peroxide is the gas generating agent and the hypohalite is sodium, potassium, lithium or calcium hypochlorite a preferred mole ratio of hypohalite to hydrogen peroxide in solution is between about 1:10 moles to 1:1, preferably it is 1:8 to 4:5 and more preferably it is 1:2 to 1:7.

A pH adjusting agent may be added to the hypohalite containing liquid such that when the first and second liquid are admixed the resulting pH of the admixture is greater than about 7.0, more preferably greater than about 7.5 and most preferably greater than about 8.0. For greatest stability of the sodium hypochlorite, storage (before mixing the two solutions) at pH greater than about 11 is preferred. pH adjusting agents include any organic or inorganic bases that increase alkalinity. Preferably, the pH adjusting agent is a carbonate, bicarbonate, hydroxides, hydroxide or oxide, or mixtures thereof. An alkaline pH adjusting agent is present in an amount from about 0 to 90 weight percent of the liquid in which it is stored, preferably about between 0.1 to 15 weight percentage.

Quaternary ammonium compounds generally have the following formula R_{I}R₂R₃R₄N⁺X⁻. Depending on the nature of the R groups, the anion, and the number of quaternary nitrogen atoms present, the antimicrobial QAC are typically classified as mono alkyl trimethyl ammonium compounds, mono alkyl dimethylbenzyl ammonium salts, dialkyl dimethyl ammonium salts, heteroaromatic ammonium salts, polysubstituted quaternary ammonium salts, bisquaternary ammonium salts or polymeric ammonium salts. Examples of mono alkyl trimethyl ammonium salts include cetyl trimethyl ammonium bromide (CTAB); alkyl trimethyl ammonium chloride; alkyl aryl trimethyl ammonium chloride; cetyl dimethyl ethyl ammonium bromide. Examples of mono alkyl dimethyl benzyl ammonium salts include alkyl dimethyl benzyl ammonium chlorides; dodecyl dimethyl 3,4 dichlorobenzyl ammonium chloride; and mixtures of alkyl dimethyl benzyl and alkyl dimethyl substituted benzyl (ethyl benzyl) ammonium chlorides. Examples of dialkyl dimethyl ammonium salts include didecyl dimethyl ammonium halides and octyl dodoceyl dimethyl ammonium chlorides. Examples of heteroaromatic ammonium salts include cetylpyridinium halide (CPC); 1-[3-chloroallyl]-3,5,7-triaza-1-azoniaadamantane; alkyl-isoquinoliniumm bromide and alkyldimethylnaphthylmethyl ammonium chloride. Examples of poly-substituted quaternary ammonium compounds include alkyl dimethyl benzyl ammonium saccharinate and alkyl dimethylethylbenzyl ammonium cycloheylsulfamate. Examples of bis-quaternary ammonium salts include 1,10-bis(2-mthyl-4-aminoquinolinium chloride)-decane; b1,6-Bis[1-methyl-3-(2,2,6-trmethyl cyclohexyl)-propyldimethyl ammonium chloride]hexane.

Particularly suitable quaternary ammonium compounds are alkyl dimethyl benzyl ammonium chlorides, alkyl dimethyl ethylbenzyl ammonium chlorides, dialkyl dimethyl quaternary compounds or mixtures of these quaternary compounds.

Of special interest are alkyl dimethyl benzyl ammonium chlorides where the alkyl group is a mixture of 50% C14, 40% C12 and 10% C16. Another alkyl dimethyl benzyl ammonium chloride of interest is where the alkyl group is a mixture of 5% C12, 60% C14, 30% C16 and 5% C18.

Alkyl dimethyl ethylbenzyl ammonium chlorides of special interest include those where the distribution of alkyl groups are as follows 50% C12, 30% C14, 17% C16 and 3% C18. Also of interest is alkyl dimethyl ethylbenzyl ammonium chlorides where the alkyl group has the distribution of 68% C12 and 32% C14.

Dialkyl dimethyl ammonium chlorides of special interest include didcecyl dimethyl ammonium chlorides; dioctyl dimethyl ammonium chloride; didecyl dimethyl ammonium chloride and octyl decyl ammonium chloride.

Mixtures of the preferred quaternary ammonium compounds would be suitable. A broad range of quaternary ammonium compound concentrations are used. A skilled artisan can determine what effective minimal amount might suffice under a specific set of conditions, such as the amount of biofilm. Preferably, the quaternary ammonium compounds concentration range in the foam composition is about between 100 ppm and 50,000 ppm.

Either solution can be thickened, preferably with surfactant thickener. Examples of surfactants than can be used to modify the solutions include C14-C18 alkyl betaines, for example cetyl dimethyl betaine, or amine oxides, for example cetamine oxide, decylamine oxide, lauramine oxide or modified amine oxides. Additional thickeners such as polymers or gums are suitable as long as the desired foam characteristics and compatibility are obtained.

To be effective as a disinfectant, the admixture shall contain at least about 100 ppm of QAC and hydrogen peroxide, each, up to about 50,000 ppm of QAC and hydrogen peroxide, each. To be effective as a biofilm remover and disinfectant, the concentration of QAC and hydrogen peroxide preferably will be about 650 ppm to about 50,000 ppm, each. A skilled artisan would recognize that higher levels of hydrogen peroxide and QAC would still be effective as a biofilm remover and as a disinfectant. Higher levels would be suitable for example in products sold in concentrated form (e.g. meant to be diluted prior to being applied).

An effective amount of chelating agent in the foaming mixture would typically be between about 50-50,000 ppm, preferably about between 100-30,000 ppm, more preferably about 25,000 ppm. A skilled artisan would recognize specific circumstances where an even higher amount of chelating agents might be desirable, such as in the situation of very “hard” water. The methods and compositions of the invention can accommodate higher concentrations of chelating agent(s).

A number of other adjuvants known to be compatible with the first and second liquids and components thereof may be used. One such class of adjuncts is cleaning actives that act chemically, enzymatically or through physical interactions to provide cleaning. Useful compounds are acids, bases, oxidants, reductants, solvents, enzymes, thiorganic compounds, surfactants, detergents, and mixtures of thereof. Other non-cleaning active adjuncts as known in the art may also be included. These include corrosion inhibitors, chelating (sequestering) agents, dyes and fragrances.

Any number of surfactants may be used. However it is most preferred if the selected surfactant is compatible with hydrogen peroxide and QAC and is stable under alkaline conditions. Suitable surfactants include amine oxides, betaines, alkyl glucosides and nonionic surfactants. Of interest are amine oxides, betaines and alkyl glucosides. Of special interest are C14-C18 alkyl betaines like cetyl dimethyl betaine; amine oxides such as cetamine oxide, decylamine oxide and lauramine oxide; and alkyl glucosides such as decyl glucoside.

### EXAMPLE 1

Solution 1 (FB3-59-2)

The following ingredients were added to a 250 ml glass beaker and stirred with a Teflon coated magnetic stir bar until homogenous.

| | | Trade Name | |
|---|---|---|---|
| Trade Name | Chemical | Owner | Quantity (g) |
| NA | Deionized Water | NA | 39.5 |
| Barquat ™ 4250Z | Mixture of n-Alkyl | Lonza | 6.0 |
| | (C12 68%, C14 32%) | | |
| | ethylbenzyl | | |
| | ammonium chloride | | |
| | and n-Alkyl (c14 60% | | |
| | c16 30%, C12 5% | | |
| | and C18 5%) | | |
| | ammonium chloride | | |
| | (50% by weight in | | |
| | water) | | |
| M ™70 | Hydrogen Peroxide | Solvay | 4.5 |
| | (70% in water) | | |
| Glucopan ™ 425N | Decyl glucoside 48% | Cognis | 1.5 |
| | Active | Corporation | |

Solution 2

Solution 2 consisted of a 6% sodium hypochlorite solution.

When 25 ml of Solution 1 and 25 ml of Solution 2 from the above example were poured simultaneously into a 500 ml glass graduated cylinder (Pyrex No. 3042) the total volume of foam and liquid reached 325 ml of height, nearly all as foam. The volume of foam produced was at least six fold that of the combined original solutions, premix.

### EXAMPLE 2

Solution 1 (FB3-60-1)

The following ingredients were added =to a 600 ml glass beaker and stirred with a Teflon coated magnetic stir bar until homogenous.

| Trade Name | Chemical | Supplier | Quantity (g) |
|---|---|---|---|
| | Deionized Water | | 378.5 |
| B-Cap 50 | Hydrogen Peroxide | FMC | 62.1 |
| | 50% by weight in water | Corporation | |
| Barquat ™ 4250Z | Mixture of n-Alkyl | Lonza | 59.4 |
| | (C12 68%, C14 32%) | | |
| | ethylbenzyl | | |
| | ammonium chloride | | |
| | and n-Alkyl (c14 60% | | |
| | c16 30%, C12 5% | | |
| | and C18 5%) | | |
| | ammonium chloride | | |
| | (50% by weight in | | |
| | water) | | |

Solution 2 (FB3-60-2)

The following ingredients were added to a 250 ml glass beaker and stirred with a Teflon coated magnetic stir bar until homogeneous

| Chemical | Quantity (g) |
|---|---|
| Deionized Water | 50.0 |
| Sodium hypochlorite | 50.0 |
| 6% by weight in water | |

When 25 ml of Solution 1 (FB3-60-1) and 25 ml of Solution 2 (FB3-60-2) from the above example were poured simultaneously into a 500 ml glass graduated cylinder (Pyrex No. 3042) the total volume of foam and liquid reached a maximum of 220 ml of height in 30 seconds. After three minutes the foam height was 175 ml and at 5 minutes the foam height remained at approximately 150 ml. The volume of foam created was at least about three fold that of the combined volumes of the liquids, premix.

### EXAMPLE 3

Solution 1 (FB3-62-1)

The following ingredients were added to a 1000 ml glass beaker and stirred with a Teflon coated magnetic stir bar until homogenous.

| Trade Name | Chemical | Supplier | Quantity (g) |
|---|---|---|---|
| NA | Deionized Water | | 909.0 |
| B-Cap ™ 35 | Hydrogen Peroxide 35% | FMC | 71.0 |
| Hydrogen | by weight in water | Corporation | |
| Peroxide | | | |
| Barquat ™ 4250Z | Mixture of n-Alkyl | Lonza | 20.0 |
| | (C12 68%, C14 32%) | | |
| | ethylbenzyl | | |
| | ammonium chloride | | |
| | and n-Alkyl (c14 60% | | |
| | c16 30%, C12 5% | | |
| | and C18 5%) | | |
| | ammonium chloride | | |
| | (50% by weight in | | |
| | water) | | |

Solution 2 (FB3-62-2)

The following ingredients were added to a 1000 ml glass beaker and stirred with a Teflon coated magnetic stir bar until homogeneous.

| Chemical | Quantity (g) |
|---|---|
| Deionized Water | 380.0 |
| Sodium hypochlorite | 500.0 |
| 6% in water | |
| Sodium Carbonate | 120.0 |

When 25 ml of Solution 1 (FB3-62-1) and 25 ml of Solution 2 (FB3-62-2) from the above example were poured simultaneously into a 500 ml glass graduated cylinder (Pyrex No. 3042). The total volume of foam+liquid and the following characteristics were measured.

Foam Generation:

| Time (min) | Foam + Liquid Volume (ml) |
|---|---|
| 0.5 | 180 ml |
| 1.0 | 190 ml |
| 1.5 | 200 ml |
| 2.0 | 205 ml |
| 3.0 | 210 ml |
| 5.0 | 200 ml |
| 6.0 | 190 ml |
| 7.0 | 175 ml |
| 10.0 | 150 ml |
| 12.0 | 150 ml |

Solution 1 pH was measured as 6.31. Solution 2 pH was measured at 11.5.

Solution 1 had a measured hydrogen peroxide concentration of 2.54% and a measured quaternary ammonium concentration of 1.02. After Solution 1 and Solution 2 were added together on an equal volume basis the final concentration of hydrogen peroxide was expected to be 0.42%. The actual concentration of hydrogen peroxide was measured at 0.39%.

Albeit the measurements stopped after 12 minutes, the foam volume stayed at above two-fold the combined premix liquid volumes for a much longer period, at least about 20 minutes.

### EXAMPLE 4

In this example, the hypohalite has been excluded. The result of adding Solution 1 and Solution 2 would result in a mixture having the concentrations of actives (quaternary ammonium compound and hydrogen peroxide) shown in the examples above.

Solution 1 (Lot 7H085)

The following ingredients were added by adding the ingredients to a chemically compatible mixing tank and stirred until homogeneous.

| Trade Name | Chemical | Supplier | Quantity |
|---|---|---|---|
| | Deionized Water | | 70% |
| B-Cap ™35 | Hydrogen Peroxide 35% | FMC Corporation | 18% |
| Hydrogen | by weight in water | | |
| Peroxide | | | |
| Barquat ™ 4250Z | Mixture of n-Alkyl | Lonza | 12% |
| | (C12 68%, C14 32%) | | |
| | ethylbenzyl | | |
| | ammonium chloride | | |
| | and n-Alkyl (c14 60% | | |
| | c16 30%, C12 5% | | |
| | and C18 5%) | | |
| | ammonium chloride | | |
| | (50% by weight in | | |
| | water) | | |

Solution 2 (Lot 88007)

The following ingredients were added to a chemically compatible mixing tank and stirred until homogeneous

| Trade Name | Chemical | Supplier | Quantity |
|---|---|---|---|
| NA | Deionized Water | | 75% |
| NA | Potassium Carbonate | | 6% |
| Versene 100 | EDTA 39% in water | Dow Chemical | 13% |

To demonstrate the ability of the claimed product to act as a disinfectant, the microbial efficacy of the mixture of Example 4 was tested using Use Dilution Method, AOAC Official Methods of Analysis, 1990 and according to guidelines specified by the EPA in DIS/TSS-1 Efficacy Data Requirements and DIS/TSS-2 Efficacy Requirements. The test organisms were Staphylococcus aureus (ATCC 6538), Salmonella choleraesuis (ATCC 10708) and Pseudomonas aeruginosa (ATCC 15422).

100 ml of Solution 1 above was added to 100 ml of Solution 2 and diluted with 800 ml of 400 ppm AOAC Synthetic Hard Water. The diluted samples were then exposed to the above organisms using the AOAC Use Dilution method. After 10 minutes of exposure, no growth of Staphylococcus aureus, Salmonella choleraesuis or Pseudomonas aeruginosa in any of the 60 primary or secondary subculture tubes was observed.

### EXAMPLE 5

Solution 1

The following ingredients were added to a chemically compatible mixing tank and stirred until homogeneous.

| Trade Name | Chemical | Supplier | Quantity |
|---|---|---|---|
| NA | Deionized Water | | 70% |
| B-Cap ™ 35 | Hydrogen Peroxide | FMC Corporation | 18% |
| Hydrogen | 35% by weight in water | | |
| Peroxide | | | |
| Barquat ™ 4250Z | Mixture of n-Alkyl | Lonza Inc. | 12% |
| | (C12 68%, C14 32%) | | |
| | ethylbenzyl | | |
| | ammonium chloride | | |
| | and n-Alkyl (c14 60% | | |
| | c16 30%, C12 5% | | |
| | and C18 5%) | | |
| | ammonium chloride | | |
| | (50% by weight in | | |
| | water) | | |

Solution 2

The following ingredients were added to a chemically compatible mixing tank and stirred until homogeneous

| Trade Name | Chemical | Supplier | Quantity |
|---|---|---|---|
| | Deionized Water | | 75% |
| | Potassium Carbonate (Armand | | 6% |
| | Products) | | |
| | Sodium Carbonate (General | | 6% |
| | Chemical Corp) | | |
| Vesene 100 | EDTA 39% by weight in water | Dow Chemical | 13% |

A stock solution was made for testing of biofilm removal by taking one part of Solution 1, one part of Solution 2 and 10 parts of synthetic hard water.

To demonstrate the removal of biofilm from hard surfaces, a study was performed in 40 clinic dental unit lines (DUWL) randomly selected from two-year-old units of the Baltimore College of Dental Surgery, Dental School, University of Maryland, Baltimore, Md. Untreated controls were used in each study to demonstrate the efficacy of the claimed formulation. The DUWL contained multi-species, natural biofilm, accumulated during the typical dental practice operating conditions. Comparisons (untreated lines were negative controls) for efficacy in removing biofilm and disinfecting the DUWL were made in dental unit water lines after overnight exposure followed by a flushing of the DUWL for two minutes with sterile water. Treatment effectiveness was assessed through measurement of planktonic bacteria in the DUWL effluent, measurement of bacterial counts in the biofilm and evaluation of the extent of removal of the biofilm matrix. The biofilm evaluation was completed by imaging treated DUWL lines by scanning electron microscopy (SEM).

A single overnight treatment of multi-species, natural biofilm from operating DUWL as described above eliminated all bacteria from the circulating water (4.5 log 10 reduction/ml) and biofilm (3.74 log 10 reduction/cm2).

### EXAMPLE 6

Solution 1 (FB3-111-1)

The following ingredients were added to a 2000 ml glass beaker and stirred with a Teflon coated magnetic stir bar until homogenous.

| Trade Name | Chemical | Supplier | Quantity (g) |
|---|---|---|---|
| NA | Deionized Water | | 1137.3 |
| B-Cap ™ 35 | Hydrogen Peroxide 35% | FMC | 88.0 |
| Hydrogen | by weight in water | Corporation | |
| Peroxide | | | |
| Barquat ™ 4250Z | Mixture of n-Alkyl | Lonza | 24.8 |
| | (C12 68%, C14 32%) | | |
| | ethylbenzyl | | |
| | ammonium chloride | | |
| | and n-Alkyl (c14 60% | | |
| | c16 30%, C12 5% | | |
| | and C18 5%) | | |
| | ammonium chloride | | |
| | (50% by weight in | | |
| | water) | | |

Solution 2 (FB3-62-2)

The following ingredients were added to a 2000 ml glass beaker and stirred with a Teflon coated Magnetic stir bar until homogeneous

| Chemical | Quantity (g) |
|---|---|
| Deionized Water | 1023.9 |
| Sodium hypochlorite (12.5%) | 384.3 |
| Sodium Carbonate | 192.1 |

251 ml of Solution 1 is poured simultaneously with 251 ml of Solution 2 into a drain pipe having an inner diameter of 2 inches and a length from the p-trap to the drain cap of approximately 20 inches. The pipe is essentially filled by foam from the p-trap to the drain cap. The foam substantially fills the pipe for 10 minutes and then it is flushed with water. The drain pipe is swabbed and the swab tested for bacterial contamination. Testing shows at least a 3 log reduction of bacteria.

In the above Examples 1-6, the amounts listed under the Quantity columns are in weight units of the chemical compound listed or as a percentage of the total weight of the solution described. The chemical compounds described as diluted in water or obtained from any particular supplier do not limit the invention to that supplier or the use of a particular concentration. An artisan skilled in the art can readily figure a different supplier, an equivalent compound not sold under the particular trademark, or the preparation of an alternative concentration of the indicated chemical as an ingredient in the liquid, rendering a final solution fully equivalent to the solution described in the examples.

All references, including publications, patent applications, patents, and website content cited herein are hereby incorporated by reference to the same extent as if each reference were individually and specifically indicated to be incorporated by reference and was set forth in its entirety herein.

The use of the terms “a” and “an” and “the” and similar referents in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.,” “such as”) provided herein, is intended merely to better illuminate the invention and does not pose a limitation on the scope of the invention, unless otherwise claimed. No language in the specification should be construed as indicating any non-claimed element as essential to the practice of the invention.

What is claimed is:

1. An in-situ foaming drain, vessel, or hard-surface disinfectant aqueous composition comprising:
at least two liquid solutions mixing in-situ and creating the foaming composition, which foaming composition comprises a quaternary ammonium compound, peroxygen, and is at an alkaline pH,
wherein, premixing, at least one of the liquid solutions comprises water and a peroxygen,
wherein, premixing, at least one of the liquid solutions comprises water, a hapohalite and an alkaline pH adjusting agent,
wherein, premixing, at least one of the mixing water solutions solution comprises a quaternary ammonium compound.

2. The composition of claim 1, where the hypohalite is a hypochlorite.

3. The composition of claim 2, where the hypochlorite is sodium hypochlorite.

4. The composition of claim 1, where the peroxygen compound is selected from among hydrogen peroxide, a perborate, a percarbonate, an organic peracid, an inorganic peracid or a persalt.

5. The composition of claim 4, where the peroxygen compound is hydrogen peroxide.

6. The composition of claim 1, where the pH adjusting agent is a carbonate, bicarbonate, hydroxides, or oxide.

7. The composition of claim 1, where the pH adjusting agent is a sodium salt of a carbonate, bicarbonate, hydroxides, or oxide or potassium salt of carbonate or calcium salt of oxide or hydroxide.

8. The composition of claim 1, where on a molar basis the peroxygen is in excess of the hypohalite in the solutions premixing and the foaming composition comprises about between 100 and 50,000 ppm peroxygen.

9. The composition of claim 8, where the foaming composition comprises about between 650 and 50,000 ppm peroxygen.

10. The composition of claim 1, where the quaternary ammonium compound in the foaming composition is at about between 100 ppm and 50,000 ppm.

11. The composition of claim 1, where the foaming composition comprises between about 650 ppm to 50,000 ppm of the quaternary ammonium compound and about between 650 ppm to 50,000 ppm of the peroxygen and the composition is a biofilm remover.

12. The composition of claim 1, where at least one of the two liquid solutions further comprises at least one of a chelating agent, a surfactant, a thickening agent, a perfume, a corrosive inhibitor.

13. The composition of claim 12, comprising a chelating agent.

14. The composition of claim 13, where the chelating agent is EDTA.

15. The composition of claim 12, comprising a surfactant or thickener.

16. The composition of claim 15, where the surfactant or thickener is an amine oxide, a C14-C18 alkyl betaine, an alkyl glucoside or a nonionic surfactant.

17. A container for maintaining at least two liquids separately, comprising at least a first compartment for storage of a first liquid and a second compartment for storage of a second liquid, where the first liquid comprises a hypohalite and the second container comprises a peroxygen, wherein the liquid from each container may be exuded from the respective compartment via separate delivery channels that either
allows for the mixing of the liquids in a chamber prior to the delivery of the mixture from the container, or
delivers the liquids such as no mixing of the liquids occurs prior to their exit from the container.

18. An in-situ foaming drain, vessel, or hard-surface disinfectant aqueous composition comprising:
at least two liquid solutions mixing in situ and creating the foaming composition, which foaming composition comprises a quaternary ammonium compound, and is at an alkaline pH,
wherein, premixing, at least one of the liquid solutions comprises water and a peroxygen,
wherein, premixing, at least one of the liquid solutions comprises water, a hypohalite and an alkaline pH adjusting source,
wherein, premixing, at least one of the mixing water solutions solution comprises a quaternary ammonium compound.

19. A method to prepare in-situ a foaming disinfectant and aqueous composition comprising:
providing at least one solution comprising water and a peroxygen,
providing at least one solution comprising water, a hapohalite and an alkaline pH adjusting agent,
providing at least one solution comprising water and a quaternary ammonium compound,
wherein the solution comprising peroxygen can not comprise hapohalite,
wherein the solution comprising a quaternary ammonium compound may be a separate solution or may be the same solution that comprises a peroxygen or the same solution that comprises a quaternary ammonium compound,
mixing the above solutions in a pipe, vessel or a hard surface, whereby foam is formed in-situ, and
treating to disinfect the pipe, vessel or a hard surface by exposure to the foam solution for at least two minutes, and wherein the foaming solution is at least twice the combined volumes of the liquid solutions premixing, and
wherein the foaming solution comprises a quaternary ammonium compound, peroxygen, and is at an alkaline pH, and
wherein the foaming solution disinfects the pipe, vessel or a hard surface.

20. The method of claim 19, where the hypohalite is a hypochlorite.

21. The method of claim 20, where the hypochlorite is sodium hypochlorite.

22. The method of claim 19, where the peroxygen compound is selected from among hydrogen peroxide, a perborate, a percarbonate, an organic peracid, an inorganic peracid or a persalt.

23. The method of claim 22, where the peroxygen compound is hydrogen peroxide.

24. The method of claim 19, where the pH adjusting agent is a carbonate, bicarbonate, hydroxides, or oxide.

25. The method of claim 24, where the pH adjusting agent is a sodium salt of a carbonate, bicarbonate, hydroxides, or oxide.

26. The method of claim 19, where on a molar basis the peroxygen is in excess of the hypohalite in the solutions premixing and the obtained foaming composition comprises about between 100 and 50,000 ppm peroxygen.

27. The method of claim 26, where the foaming composition comprises about between 650 and 50,000 ppm peroxygen.

28. The method of claim 1, where the quaternary ammonium compound in the foaming composition is at about between 100 ppm and 50,000 ppm.

29. The method of claim 1, where the foaming composition comprises between about 650 ppm to 50,000 ppm of the quaternary ammonium compound and about between 650 ppm to 50,000 ppm of the peroxygen and the composition is a biofilm remover.

30. The method of claim 1, where at least one of the at least two liquid solutions further comprises at least one of a chelating agent, a surfactant, a thickening agent, a perfume, a corrosive inhibitor.

31. The method of claim 30, comprising a chelating agent.

32. The method of claim 31, where the chelating agent is EDTA.

33. The method of claim 30, comprising a surfactant or thickener.

34. The method of claim 33, where the surfactant or thickener is an amine oxide, a C14-C18 alkyl betaine, an alkyl glucoside or a nonionic surfactant.
